(19) United States
(12) Patent Application Publication
(10) Pub. No.: US 2024/0252442 A1
(43) Pub. Date: Aug. 1, 2024

(54) CONTROLLED RELEASE COATING AGENT

(71) Applicant: NUTRITION & BIOSCIENCES USA 1, LLC, Rochester, NY (US)

(72) Inventors: MATTHIAS KNARR, BOMLITZ (DE); OLIVER PETERMANN, BOMLITZ (DE)

(21) Appl. No.: 18/292,990

(22) PCT Filed: Jul. 28, 2022
(86) PCT No.: PCT/EP2022/071199

§ 371 Date: Jan. 29, 2024

(30) Foreign Application Priority Data
Jul. 29, 2021 (EP) 21188471.3

(51) Int. Cl. A61K 9/48; A61K 47/36

(52) U.S. Cl. CPC A61K 9/4891; A61K 47/36

(57) ABSTRACT

The present invention relates to water-insoluble micro-dispersions comprising alginic acid, and in particular to water-insoluble micro-dispersions for the preparation of capsules and capsule films intended for controlled- or sustained release oral administration of medications, nutritional components and supplements.

### FIELD OF THE INVENTION

The present invention relates to water-insoluble micro-dispersions, and in particular to water-insoluble micro-dispersions for the preparation of capsules and capsule films intended for controlled- or sustained release oral administration of medications, nutritional components and supplements.

### BACKGROUND OF THE INVENTION

Many active pharmaceutical ingredients (APIs) have a limited absorption window in the upper gastrointestinal tract. Also, incorporating many APIs into conventional dosage forms may not only lead to a decrease in bioavailability, but may also lead to the inability to achieve extended therapeutic effect. Many techniques have been disclosed in the prior art to extend APIs residence time in the stomach. However, there is a need for improved or alternative controlled release system that can provide long-term exposure to active pharmaceutical ingredients, such as when their absorption window is limited to the upper gastrointestinal tract.

Accordingly, the development of extended release (ER) such as sustained-release (SR) or controlled-release (CR) dosage forms has become the subject of interest to many pharmaceutical companies in recent years. Some of these approaches to oral ER dosage forms include matrix hydrogel systems and various coatings and capsules containing such coatings. These are applied according to the characteristics of drug and its pharmacological action to control release of both hydrophilic and hydrophobic medicinal agents. Hydroxypropyl methyl cellulose (HPMC) and cellulose ether are widely used to control release of some drugs. Hydrophobic polymers (glycerides, ethyl cellulose (EC)) are used for other drugs.

Developing oral sustained release systems and formulations for water-soluble drugs with constant rate of release has become a challenge to the pharmaceutical technologists. Various formulation approaches in controlling the release of water-soluble drugs has been developed including sustained release coated granules, matrix tablets, multilayered matrix tablets, as well as polymer coatings on capsules.

Polymers like methyl- and ethyl cellulose or some polyacrylates are sometimes not considered natural or clean-label with sustainable sourcing, and accordingly there is a need in the art to find more “organic” alternatives to existing polymers. U.S. Pat. No. 9,492,394B2 relates to gastric resistant coating layers comprising salts of alginic acid.

### BRIEF DESCRIPTION OF THE DRAWINGS

FIG. 1. Alginic acid film (59 μm thickness) casted on a glass plate

FIG. 2. Permeation tests with paracetamol (APAP), 1 cm2, 2 mg/ml APAP in 0.1M HCl (pH 1.3), 37° C., Alginic acid Protacid F120NM, 1500 μm-Blade, 243 nm; A: 59 μm film thickness, B: 220 μm film thickness

### SUMMARY OF THE INVENTION

The present inventors have found that a firm, but flexible film can be formed from aqueous dispersions of alginic acid. It was further found that these films from aqueous dispersions of alginic acid showed controlled drug permeation over time, demonstrating that such films may be used for extended, such as controlled release drug formulations.

### DETAILED DESCRIPTION OF THE INVENTION

### Alginic Acid

Alginates, derived from, inter alia, brown seaweeds are linear, unbranched bio-polymers consisting of (1-4)-linked β-D-mannuronic acid (M) and α-L-guluronic acid (G) residues. Alginates are not random copolymers but consist of blocks of similar and alternating sequences of residues, for example, MMMM, GGGG, and GMGM. In extracted form alginate absorbs water quickly. The physical properties of alginates may depend on the relative proportion of the M and G blocks. Gel formation at neutral pH requires a calcium source to provide calcium ion to interact with G-blocks. The greater the proportion of these G-blocks, the greater the gel strength.

“Alginate” is the term usually used for the salts of alginic acid, but it can also refer to all the derivatives of alginic acid and alginic acid itself; Alginate is present in the cell walls of brown algae (Phaeophyceae sp.) as the calcium, magnesium and sodium salts of alginic acid. Dry, powdered, sodium alginate or potassium alginate may be obtained from an extraction process of this brown algae. The seaweed residue is then removed by filtration and the remaining alginate may then be recovered from the aqueous solution.

Another way to recover the alginate from the initial extraction solution is to add a calcium salt. This causes calcium alginate to form with a fibrous texture; it does not dissolve in water and can be separated from it. The separated calcium alginate is suspended in water and acid is added to convert it into alginic acid.

Alginates suitable for use in the practice of this invention will typically have a molecular weight such that they exhibit a viscosity in the range of 50-1,000 mPa·s. when measured at 1 wt % at 20° C. using Brookfield type RV (e.g. RVT, RVF, RVTDV) with Brookfield RV using the appropriate spindle for the viscosity range in question. The appropriate spindle for the viscosity determination can be readily determined by one of ordinary skill in the art, based on the equipment model and the viscosity range. In some embodiment, such alginates will exhibit a viscosity of between 30 and 600 mPas, such as between 100 and 600 mPas, or between 200 and 600 mPas when so measured. In some other embodiment, such alginates will exhibit a viscosity of between 30 and 400 mPas, such as between 30 and 300 mPas, such as between 30 and 200 mPas, or between 30 and 100 mPas when so measured. Spindle #2 can be used for viscosity measurements in the preferred viscosity range, with the above-specified equipment.

In some embodiments according to the present invention, a high G type alginate is used. A high G type alginate means that the alginate(s) employed in the practice of the present invention possess an average of at least 50 percent adjacent G units. In some embodiments the alginate will possess an average of at least 52 percent adjacent G units; and in other embodiments such alginate will possess an average of at least 55 percent or more of adjacent G units, as such higher the content of adjacent G units may result in improved product textures.

As used herein “alginic acid” refers to the acid as such. Alginic acid may also be referred to as algin, E400 or CAS Number 9005-32-7.

Alginic acid as a controlled release coating agent is of high interest as alginic acid is considered as an “organic” polymer in contrast to other controlled release coating agents like ethylcellulose or some polyacrylates. Alginic acid is insoluble in neutral water and therefore an aqueous dispersion must be prepared for coating. So far film forming has failed from alginic acid aqueous dispersions and no controlled release alginic acid films are known as prior art. This problem of forming films from plain alginic acid has been solved by the present inventors by methods and dispersions described herein.

It has been found that a microdispersion of alginic acid containing a plasticizer in the correct ratios or amounts is capable of forming an alginic acid film. The alginic film shows controlled drug permeation over time in a Franz cell diffusion test. Only a very low film thickness (59 μm) is required for a firm, but flexible film providing controlled release, such as for controlled release of zero order.

### Extended Release, Such as Controlled-Release or Sustained-Release Applications

The term “extended-release” as used herein in relation to a dosage refers to either sustained-release (SR) or controlled-release (CR) dosage forms or systems. Whereas SR dosages forms maintains drug release over a sustained period, but not at a constant rate, CR dosage forms maintains drug release over a sustained period at a nearly constant rate. SR dosage forms usually follow first order kinetics whereas CR forms follow zero order kinetics.

As used herein “enteral administration” refers to the administration of a medicament, a nutritional component or supplement via the human gastrointestinal tract. Enteral administration involves the esophagus, stomach, and/or small and large intestines (i.e., the gastrointestinal tract). Enteral administration includes oral, sublingual, and rectal administrations. In one preferred administration route the capsules according to the present invention is for oral administration, where the controlled-release properties of the water-insoluble micro-dispersion according to invention is used.

As used herein “water-insoluble micro-dispersion” refers to system in which distributed water-insoluble solid particles of alginic acid in micro size are dispersed in an aqueous solution.

As used herein microparticles as used herein refers to a plurality of particles with a size ranging from 0.01 micrometer to 1000 μm, typically from 0, 1 or 1 micrometer to 1000 micrometer, or with a median particle size in the range of 1 micrometer to 100 micrometer, or in the range of 1 micrometer to 10 micrometer. Typically, the microdispersion is characterized by its particle size distribution, such as by the median particle size or Dn50, or the Dn90, or the Dn10.

As used herein “the median particle size” or “Dn50” of the particles of the water-insoluble micro-dispersion of the invention is the diameter where 50 number percent of the particles have a smaller equivalent diameter and 50 number percent have a larger equivalent diameter. Typically, the Dn50 for the inventive micro-dispersion is up to 10 micrometers, more typically up to 9, 8, 7, 6 micrometers, and even just up to 5 micrometers. The Dn50 for the present inventive micro-dispersion is typically 1 micrometers or more, more typically 2 micrometers or more, and most typically 3 micrometers or more.

The “Dn90” of the particles of the water-insoluble micro-dispersion of the invention refers to the diameter where 90 number percent of the particles have a smaller equivalent diameter and the other 10 number percent have a larger equivalent diameter. The equivalent particle diameter is the diameter of a sphere having the same volume as the volume of a given particle. Typically, the “Dn90” is up to 20 micrometers, more typically up to 18, 16, 14, 12, 10 or 8 micrometers; and typically, up to 12 micrometers, more typically up to 10 micrometers, and most typically up to 7 micrometers. Typically, Dn90 is 1 micrometers or more, more typically 2 micrometers or more, and most typically 4 micrometers or more.

The “Dn10” of the particles of the water-insoluble micro-dispersion of the invention refers to the diameter where 10% of the powder particles have a smaller equivalent diameter and the other 90 number percent have a larger equivalent diameter. Typically, Dn10 is 50 nanometers or more, more typically 100, 200, 300, 400, 500 nanometers or more.

These particle sizes relate to the sizes of the particles of the water-insoluble micro-dispersion of the invention after redispersion and after sieving the dispersion with a 100-160 μm sieve. The particle sizes are measured by laser diffraction particle size analysis, e.g., using a Beckman Coulter laser diffraction particle size analyzer which is commercially available from Beckman Coulter, California, such as a Beckman Coulter LS13320MW.

The term “plasticizer” as used herein refers to a compound enhancing film formation used according to the present invention, which include: phthalic esters, such as dimethyl-, diethyl-, dibutyl-, and diisopropyl-phthalate; citric esters, such as triethyl-, tributyl-, acetyltriethyl- and acetyltributyl-citrate; phosphoric esters, such as triethyl-, tricresyl, and triphenyl-phosphate; alkyl lactate; glycol esters; glycerol and glycerol esters, such as glycerol triacetate also known as triacetine; sucrose esters; oils and fatty acid esters; butyl stearate; dibutyl sebacate; dibutyl tartrate; diisobutyl adipate, tributyrin; propylene glycol; a polyol, such as a sugar alcohol, such as sorbitol, mannitol, erythritol, xylitol, or glycerol (Propane-1,2,3-triol); and mixtures thereof.

As used herein a “small-molecule water-soluble drug” refers to any low molecular weight (less than 900 Da) organic compound soluble in water used as a medicament, a nutritional components or supplement.

As used herein “capsule” and “encapsulation” is used in its normal meaning referring to the capsules used in the pharmaceutical and nutraceutical industries, for the purposes of aiding in delivering medication, nutritional components, and supplements that may have a nasty or unpleasant taste or smell or being sensitive for degradation. As such enteric capsules will only disintegrate once it reaches the target of the intestine. Capsules of the present invention is also an alternative to gelatin, where it offers the pharmaceutical and nutraceutical industry an opportunity to serve the vegans or people following a strict religious diet. A “capsule film” as used herein refers to a film formed around a capsule. Accordingly, the water-insoluble micro-dispersions of the present invention may be used to coat a capsule made of other additional or alternative components, wherein the film of the water-insoluble micro-dispersions of the present invention provides for the effect of the capsule, such as the effect needed for enteric applications.

### Numbered Embodiments of the Invention

1. A water-insoluble micro-dispersion comprising
   a) dispersed particles of alginic acid in an amount of about 1% to about 10% by total weight of the micro-dispersion, and
   b) a plasticizer in an amount of about 2% to about 6% by total weight of the micro-dispersion, and
   c) water in an amount of at least about 88% to not more than about 96% by total weight of the micro-dispersion.
2. The water-insoluble micro-dispersion according to embodiment 1, wherein the dispersed particles of alginic acid is present in an amount of at least about 2%, such as at least about 3%, such as at least about 4%, such as at least about 5%, such as at least about 6%, such as at least about 7%, such as at least about 8%, such as at least about 9% by total weight of the micro-dispersion.
3. The water-insoluble micro-dispersion according to any one of embodiments 1 or 2, wherein the dispersed particles of alginic acid is present in an amount of not more than about 9%, such as not more than about 8%, such as not more than about 7%, such as not more than about 6%, such as not more than about 5%, such as not more than about 4%, such as not more than about 3%, such as not more than about 2% by total weight of the micro-dispersion.
4. The water-insoluble micro-dispersion according to any one of embodiments 1-3, wherein the plasticizer is present in an amount of at least about 3%, such as at least about 4%, such as at least about 5% by total weight of the micro-dispersion.
5. The water-insoluble micro-dispersion according to any one of embodiments 1-4, wherein the plasticizer is present in an amount of not more than about 5%, such as not more than about 4%, such as not more than about 3% by total weight of the micro-dispersion.
6. The water-insoluble micro-dispersion according to any one of embodiments 1-5, wherein water is present in an amount of at least about 89%, such as at least about 90%, such as at least about 91%, such as at least about 92%, such as at least about 93%, such as at least about 95% by total weight of the micro-dispersion.
7. The water-insoluble micro-dispersion according to any one of embodiments 1-6, wherein the water is present in an amount of not more than about 95%, such as not more than about 94%, such as not more than about 93%, such as not more than about 92%, such as not more than about 91%, such as not more than about 90%, such as not more than about 89% by total weight of the micro-dispersion.
8. The water-insoluble micro-dispersion according to any one of embodiments 1-7, wherein the median particle size, Dn50, of the powder particles is up to 4 μm, such median particle size, Dn50, being the size at which 50% of the powder particles have a smaller equivalent diameter and 50% have a larger equivalent diameter.
9. The water-insoluble micro-dispersion of embodiment 8, wherein the median particle size, Dn50, of the powder particles is up to 2 μm.
10. The water-insoluble micro-dispersion according to any one of embodiments 1-9, wherein Dn90 of the powder particles is up to 20 μm, Dn90 being the diameter where 90% of the powder particles have a smaller equivalent diameter and the other 10% have a larger equivalent diameter.
11. The water-insoluble micro-dispersion of any one of embodiments 1 to 10, wherein Dn10 of the powder particles is up to 20 nm, Dn10 being the diameter where 10% of the powder particles have a smaller equivalent diameter and the other 90 number percent have a larger equivalent diameter.
12. The water-insoluble micro-dispersion of any one of embodiments 1 to 11, wherein the plasticizer is a polyol, such as a sugar alcohol, such as sorbitol, mannitol, erythritol, xylitol, or glycerol (Propane-1,2,3-triol), or acetate esters of glycerol selected from the class consisting of the mono-, di-, and tri-acetates of glycerol, TEC (triethyl Icitrate), dibutyl sebacate, and dibutyl phthalate.
13. The water-insoluble micro-dispersion of any one of embodiments 1 to 12, wherein the alginic acid is selected from DuPont Protacid®, such as Protacid® F120NM, or Protacid AR-1112 (Kelacid NF).
14. The water-insoluble micro-dispersion of any one of embodiments 1 to 13, which water-insoluble micro-dispersion do not contain any other polymer, such as a polymer based on acrylate, such as a polymer based on ethyl acrylate and/or methyl methacrylate, or a polymer based on cellulose, such as methyl- and ethyl cellulose.
15. A method for the preparation of a water-insoluble micro-dispersion comprising the steps of combining dispersed particles of alginic acid in an amount of about 1% to about 10% by total weight of the micro-dispersion with a plasticizer from about 2% to about 6% by total weight of the micro-dispersion, and water in an amount of at least about 88% to not more than about 96% by total weight of the micro-dispersion.
16. A process for coating a dosage form comprising the steps of
   a) preparing the water-insoluble micro-dispersion as defined in any one of embodiments 1-14,
   b) optionally adding a further film forming aid or adjuvants to form an aqueous coating composition and
   c) coating a dosage form with the aqueous coating composition.
17. Use of a water-insoluble micro-dispersion according to any one of embodiments 1-14 for encapsulation of a drug, a nutritional or food supplement, or a combination thereof.
18. Use of according to embodiment 17 for encapsulation of a drug, a nutritional or food supplement, or a combination thereof intended for extended release, such as controlled-release applications.
19. A capsule film made from the water-insoluble micro-dispersion of any one of embodiments 1-14.
20. The capsule film according to embodiment 18, which film has a thickness of from about 20 μm to about 200 μm, such as from about 40 μm to about 150 μm, such as from about 50 μm to about 120 μm.
21. The capsule film according to any one of embodiments 19 and 20, which film is permeable to a small-molecule water-soluble drug.
22. Use of a capsule film as defined in any of embodiments 19-21 for use in a controlled release capsule.
23. A capsule comprising a capsule film of any of embodiments 19-21 and further comprising a small-molecule water soluble drug or a nutritional or food supplement or a combination thereof.
24. A process for producing capsule film comprising the steps of providing the water-insoluble micro-dispersion of any one of embodiments 1 to 14,
   pre-heating molding pins to a temperature higher than the aqueous composition, dipping the pre-heated molding pins into the water-insoluble micro-dispersion, forming a film on said molding pins by withdrawing said pins from said water-insoluble micro-dispersion, and drying the film on the molding pins.

### EXPERIMENTAL SECTION

### Examples 1

Alginic acid Protacid F120NM was sieved with a 20 μm sieve. 16.5 g of the fraction below <20 μm was milled together with 133.5 g of water in a ball mill PM400 with 400 g of 0.5 mm balls at 400 rpm for 60 min (with 20 min pause after 20 min of milling). The balls were removed from the dispersion by sieving the dispersion with a 315 μm sieve. The particle size distribution was measured with a Beckman Coulter LS13320MW (based on volume) using the universal liquid module and showed a d50 of 2 μm:
d50 2 μm
<2 μm 50%
<5 μm 77%
<10 μm 90%
<20 μm 98%
<50 μm 100%

The microdispersion was diluted with water to result a 3.5 wt-% dispersion of alginic acid and glycerine (plasticizer, 3.5 wt-%) was added.

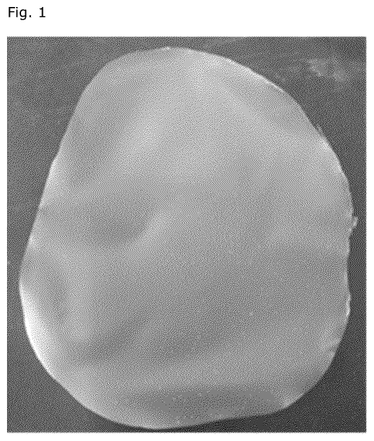

2 films were casted on a glass plate (59 μm and 220 μm film thickness) and permeation of paracetamol (APAP) was evaluated by Franz cell diffusion test (see procedure LAV 11.0) (see FIG. 1).

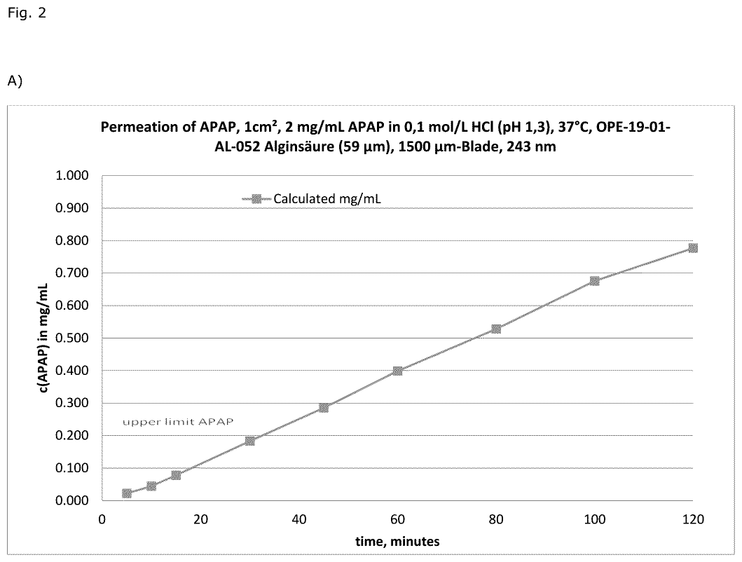

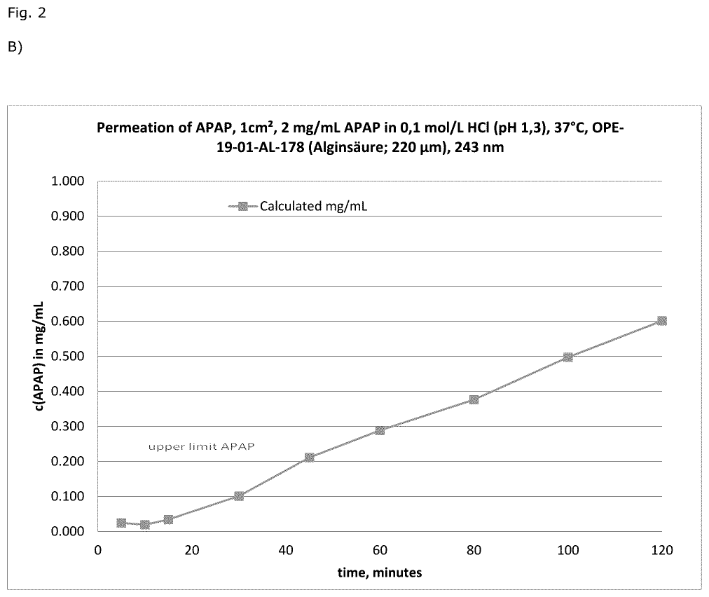

The permeation tests showed controlled release of APAP. After 120 min only 30% (220 μm film) and 40% (59 μm film) of APAP permeated trough the film. The films showed zero-order controlled release (see FIG. 2).

1. A water-insoluble micro-dispersion comprising
a) dispersed particles of alginic acid in an amount of about 1% to about 10% by total weight of the micro-dispersion, and
b) a plasticizer in an amount of about 2% to about 6% by total weight of the micro-dispersion, and
c) water in an amount of at least about 88% to not more than about 96% by total weight of the micro-dispersion.

2. The water-insoluble micro-dispersion according to claim 1, wherein the dispersed particles of alginic acid are present in an amount of as at least about 9% by total weight of the micro-dispersion.

3. The water-insoluble micro-dispersion according to claim 1, wherein the dispersed particles of alginic acid are present in an amount of not more than about 2% by total weight of the micro-dispersion.

4. The water-insoluble micro-dispersion according to claim 1, wherein the plasticizer is present in an amount of at least about 5% by total weight of the micro-dispersion.

5. The water-insoluble micro-dispersion according to claim 1, wherein the plasticizer is present in an amount of not more than about 3% by total weight of the micro-dispersion.

6. The water-insoluble micro-dispersion according to claim 1, wherein water is present in an amount of at least about 95% by total weight of the micro-dispersion.

7. The water-insoluble micro-dispersion according to claim 1, wherein the water is present in an amount of not more than about 89% by total weight of the micro-dispersion.

8. The water-insoluble micro-dispersion according to claim 1, wherein the median particle size, Dn50, of the powder particles is up to 4 μm, such median particle size, Dn50, being the size at which 50% of the powder particles have a smaller equivalent diameter and 50% have a larger equivalent diameter.

9. The water-insoluble micro-dispersion of claim 8, wherein the median particle size, Dn50, of the powder particles is up to 2 μm.

10. The water-insoluble micro-dispersion according to claim 1, wherein Dn90 of the powder particles is up to 20 μm, Dn90 being the diameter where 90% of the powder particles have a smaller equivalent diameter and the other 10% have a larger equivalent diameter.

11. The water-insoluble micro-dispersion of claim 1, wherein Dn10 of the powder particles is up to 20 nm, Dn10 being the diameter where 10% of the powder particles have a smaller equivalent diameter and the other 90 number percent have a larger equivalent diameter.

12. The water-insoluble micro-dispersion of claim 1, wherein the plasticizer is a polyol or acetate esters of glycerol selected from mono-, di-, and tri-acetates of glycerol, TEC (triethyl lcitrate), dibutyl sebacate, and dibutyl phthalate.

13-14. (canceled)

15. A method for the preparation of a water-insoluble micro-dispersion comprising combining dispersed particles of alginic acid in an amount of from about 1% to about 10% by total weight of the micro-dispersion with a plasticizer of from about 2% to about 6% by total weight of the micro-dispersion, and water in an amount of at least about 88% to not more than about 96% by total weight of the micro-dispersion.

16. A process for coating a dosage form comprising:
a) preparing the water-insoluble micro-dispersion of claim 1, and
b) coating a dosage form with the water-insoluble micro-dispersion.

17. A process for preparing an encapsulated drug, nutritional supplement or food supplement, wherein the process comprises encapsulating a drug, nutritional supplement or food supplement in Use of a water-insoluble micro-dispersion according to claim 1.

18. (canceled)

19. A capsule film, wherein the capsule film comprises a dried water-insoluble micro-dispersion of claim 1.

20. The capsule film according to claim 19, wherein the film has a thickness of from about 50 μm to about 120 μm.

21. (canceled)

22. A capsule of claim 23, wherein the capsule is a controlled release capsule.

23. A capsule, wherein the capsule comprises:
a capsule film of claim 19, and
a small-molecule water soluble drug or a nutritional or food supplement or a combination thereof.

24. A process for producing a capsule film, wherein the process comprises:
providing the water-insoluble micro-dispersion of claim 1,
pre-heating molding pins to a temperature higher than the aqueous composition,
dipping the pre-heated molding pins into the water-insoluble micro-dispersion,
forming a film on said molding pins by withdrawing said pins from said water-insoluble micro-dispersion, and
drying the film on the molding pins.
